(19) United States
(12) Patent Application Publication
(10) Pub. No.: US 2009/0053272 A1
(43) Pub. Date: Feb. 26, 2009

(54) METHOD FOR PRODUCING POLYMER NANOPARTICLES

(75) Inventor: Gerhard Wagenblast, Wechenheim (DE)

Correspondence Address: CONNOLLY BOVE LODGE & HUTZ, LLP, P O BOX 2207, WILMINGTON, DE, 19899, US

(73) Assignee: BASF SE, LUDWIGSHAFEN (DE)

(21) Appl. No.: 12/282,897

(22) PCT Filed: Mar. 13, 2007
(86) PCT No.: PCT/EP07/52320

§ 371 Date: Sep. 15, 2008

(30) Foreign Application Priority Data
May 10, 2005 (DE) 10-2005-021-535.1
May 10, 2005 (DE) 10-2005-021-557.2
Oct. 5, 2005 (DE) 10-2005-021-560-2

(51) Int. Cl. A61K 9/14; A61K 8/02; A01N 25/12

(52) U.S. Cl. 424/401; 424/405; 424/501

(57) ABSTRACT

A process for the preparation of polymeric nanoparticles, in which an emulsion of monomers and additional components is produced in a nonsolvent and subsequently illuminated, makes it possible to prepare polymeric nanoparticles which comprise, in a desired concentration, an effect substance, for example a dye, and/or an active substance, for example a herbicide.

The present invention refers to a process for the preparation of polymeric nanoparticles in which the polymeric product is obtained, starting from polymerizable monomers, using light energy. It furthermore relates to the polymeric nanoparticles which can comprise effect substances and/or active substances and also to their use.

Nanoparticles are playing an increasing role in many areas of industrial production. From chip technology through rubber production to medicine and cosmetics, nanoparticles are finding advantageous possible uses because of their exceptional material properties. Generally, nanoparticles exhibit distinctly different physical and chemical properties than their coarse-grained analogs. This results in special application possibilities since, with a small particle volume, a very high surface area and also a chemically variable surface can be made available. Thus, for example, with polymeric nanoparticles, it is possible for the energies occurring in the material to be strongly dispersed, which has resulted, for example in the field of the rubber industry, in an increased elasticity of tires and a reduced rolling resistance. Polymeric nanoparticles can, however, also be usefully employed in the fields of textiles and paper manufacture.

U.S. Pat. No. 6,403,672 describes a process for the preparation of polymeric nanoparticles in which monomers are illuminated in a nonaqueous solvent.

It is an object of the present invention to make available a process for the preparation of small polymeric particles, in particular of nanoparticles, in which a polymeric product with a nanoparticulate structure can be cost-effectively prepared using light energy using polymerizable monomers, in particular monomers which can be polymerized by introducing light energy, and, if appropriate, additional auxiliaries.

A subject matter of the invention is a process for the preparation of polymeric nanoparticles in which one or more polymerizable monomers (M) and, if appropriate, one or more dispersants (D) and/or one or more effect substances (E) and/or one or more active substances (A) are introduced into a nonsolvent (N), such as, for example, water, and the emulsion resulting therefrom (for example, by vigorous stirring) is illuminated with a suitable light source (L). In this connection, the necessary energy for the reaction is applied by the light source (L), resulting in the desired photopolymerization.

Monomeric starting materials which can typically be polymerized by light, in particular by UV light, for example polyfunctional acrylates, can be used as monomers (M). For example, mono-, di- or triacrylates (see, e.g., products of the Laromer® series, for example Laromer LR 8863, an ethoxylated trimethylolpropane triacrylate; manufacturer BASF, Ludwigshafen) can be used. It is also possible to use, as a mixture, different monomers which can be polymerized by UV light. Reference is made to the literature with regard to the monomers which can be polymerized by photopolymerization.

The present invention relates in particular to a process for the preparation of polymeric nanoparticles in which one or more polymerizable monomers (M) and one or more dispersants (D) and/or one or more effect substances (E) and/or one or more active substances are introduced into a nonsolvent (N), preferably water, and the emulsion resulting therefrom is illuminated with a suitable light source (L), in particular UV light, resulting in a photopolymerization of the monomers (M).

The term “a nonsolvent” is understood to mean, in the present invention, a liquid in which the monomers exhibit, at ambient temperature and at the processing temperature, a solubility of less than 5 g/l, in particular of less than 1 g/l. Use is preferably made, as nonsolvent, of water or a mixture of water with an additional water-miscible liquid. However, it is in principle also possible to use other liquids than water or liquid mixtures as nonsolvent. The nonsolvent (N) can also comprise dissolved components, for example a dispersant (D).

In one embodiment of the invention, use is made of at least two different monomers (M) as a mixture. Use is preferably made, e.g., of two or three different acrylate monomers.

In the process for the preparation of polymeric nanoparticies, the polymerizable monomers (M) are preferably first mixed with a photoinitiator (P) and, if appropriate, one or more effect substances (E) and/or one or more active substances (A). Subsequently, this mixture is mixed with the nonsolvent. An emulsion with the nonsolvent (N) and the dispersant (D) is produced and the emulsion thus formed, also described as crude emulsion, if appropriate after producing a fine emulsion produced by introducing shear energy, is subjected to an illuminating stage with UV light. The emulsions can also comprise, in addition to the dispersant (D), a protective colloid (C).

Suitable emulsifiers are commercial dispersants or wetting agents. The emulsifiers or dispersants (D) used can be anionic, cationic and also nonionic in nature. The anionic emulsifiers include alkali metal and ammonium salts of alkyl sulfates (e.g., from C₈ to C₁₂ alkyl radical), of sulfuric acid monoesters of ethoxylated alkanols (degree of ethoxylation: from 2 to 50, alkyl radical: C₁₂ to C₁₈) and of ethoxylated alkylphenols (degree of ethoxylation: from 3 to 50, alkyl radical: C₄ to C₉), of alkylsulfonic acids (alkyl radical: C₁₂ to C₁₈) and of alkylarylsulfonic acids (alkyl radical: C₉ to C₁₈). Suitable nonionic emulsifiers are araliphatic or aliphatic nonionic emulsifiers, for example ethoxylated mono-, di- and trialkylphenols (degree of ethoxylation: from 3 to 50, alkyl radical: C₄ to C₉), ethoxylates of long-chain alcohols (degree of ethoxylation: from 3 to 50, alkyl radical: C₈ to C₃₆) and polyethylene oxide/polypropylene oxide block copolymers. Suitable examples are furthermore lignosulfonates, naphthalenesulfonic acid/formaldehyde condensates and phenol/cresol/sulfanilic acid/formaldehyde condensates.

Natural or semisynthetic protective colloids (C) which can be used according to the invention are, for example, gelatins, fish gelatins, starch or starch derivatives, such as dextrins, pectin, gum arabic, casein, caseinate, alginates, cellulose and cellulose derivatives, such as methylcellulose, carboxymethylcellulose, hydroxypropylcellulose or hydroxypropylmethylcellulose.

Synthetic protective colloids which can be used are water-soluble homo- or copolymers which can be neutral polymers, cationic polymers and anionic polymers. Complexes of polycationic and polyanionic polymers and also coacervates are also suitable.

Polymers which can be used as protective colloid are in particular polyvinylpyrrolidone, polyacrylic acid or polymethacrylic acid and copolymers thereof with a dicarboxylic anhydride of an ethylenically unsaturated C₄-C₈-carboxylic acid, such as maleic anhydride or itaconic anhydride; polyvinyl alcohol and partially saponified polyvinyl acetate; polyacrylamide and polymethacrylamide and the partially saponified derivatives thereof; polymers of monomers with a primary, secondary or tertiary amino group and the N-C₁-C₄-mono- and N,N-di-C₁-C₄-alkyl derivatives thereof and the derivatives thereof quaternized with C₁-C₄-alkylating agents; polyethylene oxides and polypropylene oxides and block copolymers thereof; polyamino acids, such as polyaspartic acid and polylysine; and condensates of phenylsulfonic acid with urea and formaldehyde and condensates of naphthalenesulfonic acid with formaldehyde.

In one embodiment of the invention, use is additionally made, in the process, in addition to polymerizable monomers, e.g. acrylate monomers, and, if appropriate, an effect substance (E) and/or, if appropriate, an active substance (A), of one or more photoinitiators (P).

Use may generally be made, as photoinitiators, of those organic compounds which absorb UV light and in this connection generate highly reactive intermediates (in particular radicals) and accordingly can initiate a photopolymerization. Suitable categories of compounds are photoinitiators of benzophenone type, of acylphosphine oxide type (such as, e.g. the commercial product Lucirin TPO from BASF, Ludwigshafen, or diphenyl(2,4,6-trimethylbenzoyl)phosphine oxide), of bisacylphosphine oxide type, photoinitiators such as the commercial Irgacure (manufacturer Ciba, Switzerland) or photoinitiators which generate benzoyl radicals (e.g. 2,2-dimethoxy-1,2-diphenylethanone).

The invention also relates to a process for the preparation of polymeric nanoparticles in which use is made of an emulsion which additionally comprises one or more effect substances (E) from the group consisting of dyes, optical brighteners, UV absorbers and pigments and/or one or more active substances (A) from the group consisting of pesticides, biocides, pharmaceuticals and fragrances. These effect substances (E) and/or these active substances (A) are preferably mixed with the monomers in a suitable mixing ratio. This organic phase can then be introduced into the aqueous phase (which already comprises, e.g., the emulsifier or the dispersant), preferably followed by mechanical mixing.

The mixing operation and the entire preparation process can in this connection be carried out continuously or noncontinuously. Preferably, a crude emulsion is first generated and this is then converted to a fine emulsion by introducing additional shear energy. In this connection, the term “fine emulsion” is understood to mean an emulsion in which the particles (or droplets) of the organic component have a mean particle size of less than 10 μm, in particular of less than 4 μm.

In the process according to the invention for the preparation of polymeric nanoparticles, a crude emulsion is frequently first prepared which comprises droplets of the monomers (and, if appropriate, of additional organic components) emulsified in the nonsolvent. The crude emulsion can be prepared in different ways.

For example, the photoinitiator (P), the effect substance (E) and/or the active substance (A) are first dissolved, emulsified or dispersed in the monomers.

The mixture of monomers and, if appropriate, photoinitiator (P), effect substance (E) and/or active substance (A) produced can be roughly emulsified under low-shear conditions (e.g., stirring).

It is particularly advantageous to carry out the preparation of the crude emulsion in the nonsolvent in a continuous process in which, in principle, an increase in temperature can also be employed. The crude emulsion thus obtained comprises relatively large droplets of the organic components in the nonsolvent. The mean particle size is in this connection generally more than 5 μm, it also being possible in particular for numerous clearly larger droplets to occur, for example with a mean particle size of more than 20 μm. The crude emulsion frequently comprises dispersants or emulsifiers and/or protective colloids. These dispersants or emulsifiers and/or protective colloids can be added to the crude emulsion at any point in time; however, they can also already be present in the nonsolvent (N) on introducing the organic components. Suitable emulsifiers are commercial dispersants or wetting agents.

The shear energy can be introduced in different ways. The crude emulsion can be treated batchwise in one stage or in individual stages or, preferably, continuously. The shear energy can be introduced by conveying the crude emulsion through a continuous emulsifying device. The term “a continuous emulsifying device” is understood to mean devices which allow continuous introduction of shear energy into an emulsion conveyed through it.

These include, for example, rotor/stator units, such as gear rim dispersing machines or colloid mills, and high-pressure homogenizers. Gear rim dispersing machines are generally devices with at least one shearing element with a stationary annular slotted stator and a slotted rotor rotating inside the stator which is mounted on a drive shaft set so as to be rotatable. Suitable gear rim dispersing machines are commercially available.

Colloid mills (see wet rotor mills) exhibit, e.g., conically shaped rotors and stators, the surface of which can be smooth or interlocked. Suitable rotor/stator units frequently comprise several shearing elements placed one after the other.

Use is particularly preferably made, in the present invention, of high-pressure homogenizers in which the crude emulsion is compressed through fine nozzles at a predetermined pressure.

The homogenizing effect is based on the production of turbulent and/or laminar flows and shear gradients resulting therefrom and also cavitation in the vicinity of large droplets, which thereby tear. The pressure difference at the nozzle is generally between 10 and 1000 bar, preferably from 20 to 300 bar. In this connection, use may be made, e.g., of ring-shaped nozzles or hole-type nozzles.

It is likewise possible to use a homogenizing head thus mentioned which can be adjustable. Hole-type nozzles are preferred, in particular those with a hole diameter of 0.3 to 0.5 mm. The fine emulsifying of the crude emulsion can also be carried out using a cascade of high-pressure homogenizers or a combination of rotor/stator units and high-pressure homogenizers. Emulsifying devices with a nozzle shaped as an annular opening which exhibits an adjustably arranged dispersing element shaped as a conical point which at least partially extends into the opening have proven to be particularly worthwhile. The dispersing element is arranged in such a way that it limits the cross section effectively available to the crude emulsion for flowing through the opening. In the event of a blockage of the opening, this can be easily cleared again by a movement of the dispersing element in the opening and/or out of it. Suitable devices are “needle valves” which are normally used as control valves for regulating the flow rate of gases or liquids.

Particularly good emulsifying results are achieved for the crude emulsions described above with a device which exhibits a preliminary region and also an expansion region connected with the preliminary region via an annular opening. A dispersing element shaped as a conical point is arranged in adjustable fashion in the preliminary region or in the expansion region, which element at least partially extends into the opening, whereby the ratio of the greatest dimension (d_{E}) of the preliminary region at right angles to the flow direction to the diameter (d_{B}) of the opening is greater than 10. By this choice of the dimension of the preliminary region at right angles to the flow direction in the ratio to the diameter of the opening, a strong acceleration in the crude emulsion flowing through immediately before the opening is achieved if the ratio of the length of the opening to the diameter of the opening is less than 1.0, in particular less than 0.6. The required operating pressure can be applied by compressing the crude emulsion to the homogenization pressure using a pump, e.g. a piston pump.

Instead of using a pump, the homogenization pressure can also be produced by supplying the feed vessel of the crude emulsion or the dye suspension with a gas, such as air or nitrogen.

In a particular embodiment, the emulsion is rendered inert just before illuminating, i.e. the oxygen content in the emulsion is markedly reduced by, e.g., blowing in inert gas (e.g., nitrogen or carbon dioxide).

In a suitable embodiment of the process according to the invention, the emulsion can also be repeatedly led through a continuous emulsifying device and then illuminated.

The invention also relates to a process for the preparation of polymeric nanoparticles in which a preemulsion is first produced by stirring and then this is treated by a process for fine emulsification with introduction of shear energy and subsequently illuminated with a suitable light source, e.g. a UV lamp, and photopolymerized. The production of the preemulsion, the processing stage of the fine emulsification and the photopolymerization can generally be carried out at temperatures of 5 to 90° C., in particular of 10 to 50° C., in particular also at ambient temperature, it also being possible for the individual stages to be carried out at different temperatures.

In one embodiment of the invention, a preemulsion is first produced in the process for the preparation of polymeric nanoparticies and then this is treated by a continuous process for fine emulsification with introduction of shear energy, the emulsified particles comprising the monomers achieving a mean size of less than 4 μm. Subsequently, the fine emulsion is illuminated with a UV light source and photopolymerized. This results in the production of small polymer particles which are finely distributed or dispersed in the nonsolvent.

The invention also relates to a process for the preparation of polymeric particles, in particular of nanoparticles, in which the entire process is carried out at a temperature of 10 to 35° C.

The invention also relates to a process in which a preemulsion is first produced and then this is converted to a fine emulsion, for example using a rotor/stator process (e.g., by a colloid mill or a gear rim using dispersing machine) or using a high-pressure homogenization process (in which the crude emulsion is compressed through fine nozzles at high pressure). This fine emulsion is preferably equally with or immediately after production illuminated with a UV light source and photopolymerized. This can be carried out by providing in the apparatus, after the nozzle or expansion chamber, an illumination section through which the emulsion is conveyed.

Through this, a coalescence of the nonpolymerized droplets to give larger droplets can be avoided or at least markedly reduced, with the result that the polymeric nanoparticles produced exhibit a more homogeneous particle size distribution. With particles comprising active substances or effect substances, this can present a considerable advantage since the rate of release of the active or effect substance is correlated with the particle size of the polymer particles. Improved slow-release formulations can thus be provided.

The present invention also relates to the use of the above-described processing stages or of the process for the preparation of polymeric nanoparticies, it also being possible for these nanoparticles to comprise one or more effect substances (E) and/or one or more active substances (A).

An additional embodiment of the invention relates to finely divided polymeric particles, in particular nanoparticles, which can be prepared according to one of the above-described processes. These polymeric particles can, e.g., comprise one or more effect substances (E) and/or one or more active substances (A). The polymeric nanoparticles preferably have a mean particle size of less than 4 μm; in particular, they exhibit a mean particle size of 0.01 μm to 3.8 μm, preferably of 0.05 to 3.0 μm.

The invention also relates to polymeric nanoparticles which comprise at least one polyacrylate and which exhibit a mean particle size of 0.05 μm to 3.0 μm.

The polymeric nanoparticles comprise, in an additional embodiment, at least one effect substance (E) from the group consisting of dyes, optical brighteners, UV absorbers and pigments. This effect substance is preferably homogeneously distributed in the polymer.

The common inorganic natural pigments (e.g. chalk) or synthetic pigments (e.g. titanium oxides) but also organic pigments can be used as pigments.

Optical brighteners, which compensate through their bluish fluorescence (complementary color) for graying and yellowing, can contribute, as effect substances, in the polymeric nanoparticles, e.g., to increasing the whiteness. Suitable here are in principle all blue-emitting fluorescent dyes, e.g. the commercially available products, e.g. Ultraphor® (BASF), Leucophor® (Clariant) or Tinopal® (Ciba) or other products from the chemical categories of the stilbenes, distyrylbiphenyls, coumarins, naphthalic acid imides and the benzoxazole and benzimidazole systems linked via double bonds.

The optical brighteners can be smuggled into the preparation process separately or together with the monomers. If an optical brightener is used as effect substance, its concentration is thus generally from 0.01 to 10%, based on the weight of the monomers.

Another subject matter of the invention is polymeric nanoparticles which comprise at least one active substance (A). The active substance can belong in particular to one of the groups of the pesticides (e.g., a fungicide or herbicide), biocides (e.g., a bactericide), pharmaceuticals and fragrances. The content of active substances can be selectively controlled in the process according to the invention and differs according to the active substance. The content is generally from 0.001 to 20% by weight, based on the amount of the monomers used. The active substance is in this connection preferably homogeneously distributed in the polymeric particles.

The following categories a1) to a15) can be mentioned, for example, as herbicides which can be formulated with the nanoparticles according to the invention:
a1) lipid biosynthesis inhibitors;
a2) acetolactate synthase inhibitors (ALS inhibitors);
a3) photosynthesis inhibitors;
a4) protoporphyrinogen IX oxidase inhibitors;
a5) bleacher herbicides;
a6) enolpyruvyl shikimate 3-phosphate synthase inhibitors (EPSP inhibitors);
a7) glutamine synthetase inhibitors;
a8) 7,8-dihydropteroate synthase inhibitors (DHP inhibitors);
a9) mitosis inhibitors;
a10) inhibitors of the synthesis of long-chain fatty acids (VLCFA inhibitors);
a11) cellulose biosynthesis inhibitors;
a12) decoupler herbicides;
a13) auxin herbicides;
a14) auxin transport inhibitors;
a15) Herbicides from the group consisting of benzoylprop, flamprop, flamprop-M, bromobutide, chlorflurenol, cinmethylin, methyldymuron, etobenzanid, fosamine, metam, pyributicarb, oxaziclomefone, dazomet, triaziflam, methyl bromide and endothal.

Use is preferably made, from these categories a1) to a15), as active substance, of:
a1) chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, trifop, alloxydim, butroxydim, clethodim, cloproxydim, cycloxydim, profoxydim, sethoxydim, tepraloxydim, tralkoxydim, butylate, cycloate, diallate, dimepiperate, EPTC, esprocarb, ethiolate, isopolinate, methiobencarb, molinate, orbencarb, pebulate, prosuifocarb, sulfallate, thiobencarb, tiocarbazil, triallate, vernolate, benfuresate, ethofumesate, bensulide, pinoxaden.
a2) amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid, pyrithiobac, flucetosulfuron, orthosulfamuron, pyrimisulfan.
a3) atraton, atrazine, ametryn, aziprotryn, cyanazine, cyanatryn, chlorazine, cyprazine, desmetryn, dimethametryn, dipropetryn, eglinazine, ipazine, mesoprazine, methometon, methoprotryne, procyazine, proglinazine, prometon, prometryn, propazine, sebuthylazine, seebumeton, simazine, simeton, simetryn, terbumeton, terbuthylazine, terbutryn, trietazine, ametridione, amibuzin, hexazinone, isomethiozin, metamitron, metribuzin, bromacil, isocil, lenacil, terbacil, brompyrazon, chloridazon, dimidazon, desmedipham, phenisopham, phenmedipham, phenmedipham-ethyl, benzthiazuron, buthiuron, ethidimuron, isouron, methabenzthiazuron, monisouron, tebuthiuron, thiazafluron, anisuron, buturon, chlorbromuron, chloreturon, chlorotoluron, chloroxuron, difenoxuron, dimefuron, diuron, fenuron, fluometuron, fluothiuron, isoproturon, linuron, methiuron, metobenzuron, metobromuron, metoxuron, monolinuron, monuron, neburon, parafluron, phenobenzuron, siduron, tetrafluron, thidiazuron, cyperquat, diethamquat, difenzoquat, diquat, morfamquat, paraquat, bromobonil, bromoxynil, chloroxynil, iodobonil, ioxynil, amicarbazone, bromofenoxim, flumezin, methazole, bentazon, propanil, pentanochlor, pyridate, pyridafol.
a4) acifluorfen, bifenox, chlomethoxyfen, chlornitrofen, ethoxyfen, fluorodifen, fluoroglycofen, fluoronitrofen, fomesafen, furyloxyfen, halosafen, lactofen, nitrofen, nitrofluorfen, oxyfluorfen, fluazolate, pyraflufen, cinidon-ethyl, flumiclorac, flumioxazin, flumipropyn, fluthiacet, thidiazimin, oxadiazon, oxadiargyl, azafenidin, carfentrazone, sulfentrazone, pentoxazone, benzfendizone, butafenacil, pyracionil, profluazol, flufenpyr, flupropacil, nipyraclofen, etnipromid, bencarbazone.
a5) metflurazon, norflurazon, flufenican, diflufenican, picolinafen, beflubutamid, fluridone, flurochloridone, flurtamone, mesotrione, sulcotrione, isoxachlortole, isoxaflutole, benzofenap, pyrazolynate, pyrazoxyfen, benzobicyclon, amitrole, clomazone, aclonifen, 4-(3-trifluoromethyl-phenoxy)-2-(4-trifluoromethylphenyl)pyrimidine, see EP-A 723960, topramezone, 4-hydroxy-3-{[2-methyl-6-(trifluoromethyl)-3-pyridinyl]carbonyl}bicyclo[3.2.1]oct-3-en-2-one, known from WO 00/15615, 4-hydroxy-3-{[2-(2-methoxyethoxy)methyl-6-(trifluoromethyl)-3-pyridinyl]carbonyl}bicylo[3.2.1]oct-3-en-2-one, see WO 01/94339, 4-hydroxy-3-[4-(methylsulfonyl)-2-nitrobenzoyl]bicyclo[3.2.1]oct-3-en-2-one, see EP-A 338992, 2-[2-chloro-4-(methylsulfonyl)-3-[(2,2,2-trifluoroethoxy)methyl]-benzoyl]-3-hydroxy-2-cyclohexen-1-one (see DE 19846792), pyrasulfotole.
a6) glyphosate;
a7) glufosinate und bilanafos.
a8) asulam.
a9) benfluralin, butralin, dinitramine, ethalfluralin, fluchloralin, isopropalin, methalpropalin, nitralin, oryzalin, pendimethalin, prodiamine, profluralin, trifluralin, amiprofos-methyl, butamifos, dithiopyr, thiazopyr, propyzamide, tebutam, chlorthal, carbetamide, chlorbufam, chlorpropham, propham.
a10) acetochlor, alachlor, butachlor, butenachlor, delachlor, diethatyl, dimethachlor, dimethenamid, dimethenamid-P, metazachlor, metolachlor, S-metolachlor, pretilachlor, propachlor, propisochlor, prynachlor, terbuchlor, thenylchlor, xylachlor, allidochlor, CDEA, epronaz, diphenamid, napropamide, naproanilide, pethoxamid, flufenacet, mefenacet, fentrazamide, anilofos, piperophos, cafenstrole, indanofan, tridiphane.
a11) dichlobenil, chlorthiamid, isoxaben, flupoxam.
a12) dinofenate, dinoprop, dinosam, dinoseb, dinoterb, DNOC, etinofen, medinoterb.
a13) clomeprop, 2,4-D, 2,4,5-T, MCPA, MCPA-thioethyl, dichlorprop, dichlorprop-P, mecoprop, mecoprop-P, 2,4-DB, MCPB, chloramben, dicamba, 2,3,6-TBA, tricamba, clopyralid, fluroxypyr, picloram, triclopyr, benazolin, aminopyralid;
a14) naptalam, diflufenzopyr.
a15) benzoylprop, flamprop, flamprop-M, bromobutide, chlorflurenol, cinmethylin, methyidymron, etobenzanid, fosamine, metam, pyributicarb, oxaziclomefone, dazomet, triaziflam, methyl bromide, endothal.

With regard to the herbicides which can be used according to the invention as active substance, reference is made to “Farm Chemicals Handbook 2000 Vol. 86, Meister Publishing Company, 2000” and to “W. H. Ahrens, Herbicide Handbook, 7^{th} Edition, Weed Science Society of America, 1994”.

Mention can be made, as fungicides which can be formulated in the nanoparticles according to the invention, for example, of:

Strobilurins, such as, for example, azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho(2,5-dimethylphenyloxymethyl)phenyl)-3-methoxyacrylate;

Carboxamides, such as, e.g.,
a) Carboxanilides: benalaxyl, benodanil, boscalid, carboxin, mepronil, fenfuram, fenhexamide, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-(trifluoromethyl)biphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide, N-(2-cyanophenyl)-3,4-dichloroisothiazole-5-carboxamide;
b) carboxylic acid morpholides: dimethomorph, flumorph;
c) benzamides: flumetover, fluopicolide(picobenzamid)zoxamide;
d) other carboxamides: carpropamid, diclocymet, mandipropamid, N-(2-(4-[3-(4-chlorophenyl)prop-2-ynyloxy]-3-methoxyphenyl)ethyl)-2-methylsulfonylamino-3-methylbutyramide, N-(2-(4-[3-(4-chlorophenyl)prop-2-ynyloxy]-3-methoxyphenyl)ethyl)-2-ethylsulfonylamino-3-methylbutyramide;

Azoles, such as, e.g.,
a) triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
b) imidazoles: cyazofamid, imazalil, pefurazoate, prochloraz, triflumizole;
c) benzimidazoles: benomyl, carbendazim, fuberidazole, thiabendazole;
d) others: ethaboxam, etridiazole, hymexazole;

Nitrogen-comprising heterocyclyl compounds, such as, e.g.,
a) pyridines: fluazinam, pyrifenox, 3-[5-(4-chlorophenyl)-2,3-dimethylisoxazolidin-3-yl]-pyridine;
b) pyrimidines: bupirimate, cyprodinil, ferimzone, fenarimol, mepanipyrim, nuarimol, pyrimethanil;
c) piperazines: triforine;
d) pyrroles: fludioxonil, fenpiclonil;
e) morpholines: aidimorph, dodemorph, fenpropimorph, tridemorph;
f) dicarboximides: iprodione, procymidone, vinclozolin;
g) others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindol-1-sulfonyl)-[1,2,4]triazole-1-sulfonamide;

Carbamates and dithiocarbamates, such as, e.g.,
a) dithiocarbamates: ferbam, mancozeb, maneb, metiram, metam, propineb, thiram, zineb, ziram;
b) carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethylsulfonyl)but-2-yl)carbamate;

Other fungicides, such as, e.g.,
a) guanidines: dodine, iminoctadine, guazatine;
b) organometallic compounds: fentin salts;
c) sulfur-comprising heterocyclyl compounds: isoprothiolane, dithianon;
d) organophosphorus compounds: edifenphos, fosetyl, fosetyl-aluminum, iprobenfos, pyrazophos, tolclofos-methyl, phosphorous acid and its salts;
e) organochlorine compounds: thiophanate-methyl, chlorothalonil, dichlofluanid, tolylfluanid, flusulfamide, phthalide, hexachlorobenzene, pencycuron, quintozene;
f) nitrophenyl derivatives: binapacryl, dinocap, dinobuton;
g) others: spiroxamine, cyflufenamid, cymoxanil, metrafenon.

Mention may be made, as biocides which can be formulated with the nanoparticles according to the invention, for example, of various commercial bactericides or algicides. Biocides are used in many fields and are used for combating bacteria, fungi or algae. Use is preferably made of organic biocides as active substance according to the invention. Examples of these substances are chloroisocyanurates, quaternary ammonium compounds (quats), hydantoins, isothiazolinones, parabens, triclosan, 2-bromo-2-nitropropane-1,3-diol, phenoxyethanol or hexahydrotriazines.

In a preferred embodiment, organic biocides from the group consisting of the isothiazolin-3-ones are used. The biocidal active substance is present in the organic mixture of the monomers preferably in an amount of 0.0001 to 10% by weight, preferably of 0.001 to 1.5% by weight, in each case based on the weight of the monomers.

The use is known of compounds from the family of the 3-isothiazolin-3-ones as biocidal component in different materials. This family includes very effective biocides with a sometimes different profile of action. Combinations of different 3-isothiazolin-3-ones or also of one or more 3-isothaziolin-3-ones with other well known biocidal active substances are also often used. Additional examples of biocidal components are listed in WO 1999/08530, EP-A 0457435, EP-A 0542721 and WO 2002/17716.

Standard substances which exert an olfactory attraction, for example fragrant odoriferous substances which are used in the perfumery field (e.g., vanillin or citral), can be used as fragrances in the process according to the invention. The use of odoriferous substances in the nanoparticles is of particular interest for household products and the cosmetics industry.

Use may be made, as pharmaceutical active substances, of the most varied pharmaceuticals, e.g. analgesics (such as ibuprofen), antidiabetics, HMG-CoA reductase inhibitors, cholesterol resorption inhibitors, bile acid sorption inhibitors, antioxidants, antibiotics, antihypertensives, oncological active substances and others.

There are different preferred alternative forms of the preparation process according to the invention.

At the introduction of the polymerizable monomers into the aqueous phase, it has proven to be advantageous to add additional auxiliaries. For example, use may be made of one or more dispersants or emulsifiers. These have the object, inter alia, of resulting in an occupation of interfaces between organic and aqueous phase which is fast and as complete as possible.

Use is preferably made, as dispersants, for example, of components from the group consisting of the polyethylene glycol ethers (such as, for example, the commercial product Lutensol® TO-8, manufacturer BASF, Ludwigshafen). The dispersant or dispersants are generally used in an amount of 1 to 20% by weight, in particular of 2 to 10% by weight, based on the monomers used.

In a preferred embodiment of the invention, one or more photoinitiators (P) are additionally used, in addition to the polymerizable monomers (M). Use may be made, as photoinitiator, for example, of an acylphosphine oxide (such as, for example, the commercial product Lucirin® TPO, manufacturer BASF). The photoinitiator is generally added in an amount of 0.2 to 5% by weight, in particular of 0.5 to 3% by weight, based on the monomer used.

In an additional embodiment of the invention, an emulsion is first prepared which, in addition to the polymerizable monomer (M) and, if appropriate, other auxiliaries in the nonsolvent (N), preferably water, comprises an additional effect substance (E). These effect substances (E) are, for example, dyes (such as, for example, fluorescent or NIR dyes), optical brighteners, UV absorbers or pigments.

Use is preferably made, in the preparation of polymeric nanoparticles, of at least two different monomers (M), through which the physical properties of the polymeric product can be more precisely “tailored”.

The polymerizable monomers (M) are preferably used together with the nonsolvent (N) water. In the process for the preparation of polymeric nanoparticles, a preemulsion is preferably first produced by mechanical stirring and this is then treated using common fine emulsification processes and subsequently illuminated with a suitable light source for a period of time of approximately 0.5 second to 3 minutes and photopolymerized.

They can comprise, as effect substances (E), e.g., one or more (e.g. two) components from the group consisting of dyes, optical brighteners, UV absorbers and pigments.

In the implementation of the process according to the invention, the effect substances (E) are generally physically incorporated. The structure of the polymeric nanoparticle can be selectively influenced according to the type of effect substance (E) used. Several effect substances (E) can also be used together.

A mini-emulsion of oil in water is generally produced by the incorporation of the polymerizable monomer and of the auxiliaries in the nonsolvent. This can then be directly illuminated with a suitable light source (L), such as, for example, a UV lamp. In this connection, a spontaneous photopolymerization of the numerous “small oil droplets” occurs. The result is a dispersion of solid drop-shaped polymer particles. However, it is also possible to carry out the process according to the invention in a continuous process.

By way of example, a “preemulsion” of the individual components (M), (D), (N), (P) and/or (E) and/or (A) is produced in a large stirred vessel. This preemulsion is then converted to a “fine emulsion” using known processes of fine emulsification technology (such as, for example, use of ultrasound, high-pressure emulsifications, rotor/stator processes). The photopolymerization can subsequently be carried out by treatment of the fine emulsion with a suitable light source (L).

A UV emitter, for example an Hg lamp, a metal-doped Hg lamp, a xenon lamp or an excimer emitter, serves in particular as light source.

The polymeric nanoparticles prepared according to the process according to the invention can be put to numerous uses. For example, they can be used as light-stability agents for plastics or in cosmetics and dermatology for protecting the skin from ultraviolet light, for example if a UV absorber is used as effect substance (E). Use is also possible in the plastics and paper industry, e.g. with brightening nanoparticies (which, for example, comprise an optical brightener as effect substance), and as markers, for example the production of banknotes or forgery-proof documents.

When active substances are used, the possibility arises of preparing polymeric particles which only gradually give off the active substance (“controlled release formulations”).

This is particularly of importance in the pharmaceutical field and for plant protection compositions.

The invention is more fully explained by the following examples, without being limited to these.

### EXAMPLE 1

### 1a) Preparation of Nanoparticies of Polyether Polyacrylate

Laromer 8863 (an ethoxylated trimethylolpropane triacrylate, manufacturer: BASF AG) was used as monomer and Lucirin TPO (an aromatic acylphosphine oxide, manufacturer: BASF AG, 1.0% by weight, based on the weight of the monomers) was used as photoinitiator. In a first stage, 2 g of the monomers were mixed with the photoinitiator. In a second vessel, the dispersant Lutensol TO-8 (an oxo alcohol ethoxylate, manufacturer: BASF AG; 4% by weight, based on the amount of monomers) was added to 18 g of water.

Subsequently, the mixture of monomers and photoinitiator was added with stirring to the aqueous mixture with the dispersant. The crude emulsion thus obtained could be converted to a fine emulsion by application of ultrasound (“Sonifier 250” ultrasonic device manufactured by Branson, 150 W) for 10 minutes.

This fine emulsion was then subjected to UV illumination for 2 minutes with a gallium-doped mercury lamp, resulting in the polymerization of the monomers.

The polymer particles formed were, after the polymerization initiated by the UV illumination, centrifuged off and dried Nanoparticles in the form of small polyacrylate beads which adhere to one another were obtained. These were examined by electron microscopy. In this connection, most of the particles showed a diameter of 0.2 to 0.8 μm; individual particles had a diameter of 0.8 to 2 μm.

### 1b) Preparation of Nanoparticles of Laromer PO84F

A mixture is produced, analogously to example 1a, starting from 2 g of the monomer component Laromer PO84F (manufacturer: BASF AG) and the photoinitiator Lucirin TPO (an aromatic acylphosphine oxide, manufacturer: BASF AG, 1.0% by weight, based on the weight of the monomers). In a second vessel, the dispersant Lutensol TO-8 (an oxo alcohol ethoxylate, manufacturer: BASF AG; 5% by weight, based on the amount of monomers) is added to 18 g of water.

Subsequently, the mixture of monomers and photoinitiator is added, with intensive stirring, to the aqueous mixture with the dispersant. The crude emulsion thus obtained can be subjected to high-pressure emulsification (50 bar, 25° C., nozzle diameter 0.3 mm) and can be converted to a fine emulsion. This fine emulsion is then directly subjected, on leaving the micronization chamber, to UV illumination with a mercury lamp, resulting in the photopolymerization of the monomers. The polymer particles are nanoparticles with diameters of approximately 0.2 to 0.8 μm.

### 1c) Preparation of Nanoparticles of Laromer 8987

A mixture is produced, analogously to example 1a, starting from 1 g of the monomer component Laromer 8987 (manufacturer: BASF AG) and the photoinitiator Lucirin TPO (an aromatic acylphosphine oxide with a UV absorption maximum of approximately 380 nm; manufacturer: BASF AG, 1.0% by weight, based on the weight of the monomers). In a second vessel, the dispersant Lutensol TO-8 (an oxo alcohol ethoxylate, manufacturer: BASF AG; 3% by weight, based on the amount of monomers) is added to 18 g of water.

Subsequently, the mixture of monomers and photoinitiator is added, with stirring, to the aqueous mixture with the dispersant. The crude emulsion thus obtained can be subjected to high-pressure emulsification and can be converted to a fine emulsion. This fine emulsion is then directly subjected, on leaving the micronization chamber, to UV illumination with a mercury lamp (150 watt), resulting in the photopolymerization of the monomers. The polymer particles are nanoparticies with diameters of approximately 0.2 to 0.8 μm.

### EXAMPLE 2

2a) Preparation of Nanoparticles using a Fluorescent Dye

Nanoparticles were prepared analogously to the preparation in example 1a with additional use of 2% by weight, based on the weight of the monomers, of the commercial fluorescent dye Lumogen-F-Red 300 (manufacturer BASF). The dye was in this connection added to the mixture of monomers and photoinitiator and this mixture was then added to a vessel comprising the aqueous phase. Polymeric nanoparticles which are homogeneously “penetrated” by the effect substance were obtained. They can be used, e.g., for the preparation of a fluorescent paint or as marker.

2b) Preparation of Nanoparticies using a Fluorescent Dye

Nanoparticles can be prepared analogously to the preparation in example 2a additionally using 4% by weight, based on the weight of the monomers, of the commercial fluorescent dye Lumogen-F-Red 300. The dye can in this connection be added to the mixture of monomers and photoinitiator and this mixture can then be added to a vessel comprising the aqueous phase. Polymeric nanoparticles which are homogeneously “penetrated” by the effect substance, but with more of it, are obtained. They can be used, e.g. for the preparation of a fluorescent paint.

2c) Preparation of Nanoparticles using a Fluorescent Dye

Nanoparticles are prepared analogously to the preparation in example 1c additionally using 2% by weight, based on the weight of the monomers, of the commercial fluorescent dye Palanil fluorescent red G (manufacturer: Dystar, Leverkusen). The dye is in this connection added to the mixture of monomers and photoinitiator and this mixture is then added to a vessel comprising the aqueous phase. Polymeric nanoparticles which are homogeneously “penetrated” by the effect substance are obtained.

### EXAMPLE 3

3a) Preparation of Nanoparticles using an Optical Brightener as Effect Substance

It is possible, in the preparation of polymeric nanoparticles with optical brighteners, to use the brighteners alone or as mixtures. The optical brighteners are generally well known and commercially available products. They are, for example, described in Ullmann's Encyclopedia of Industrial Chemistry, 5th edition, Vol. A18, pp. 156-161, or can be obtained according to the methods mentioned therein.

Use is preferably made of one or more optical brighteners from the category of the coumarins, naphthalimides and styryl compounds, in particular of the cyano-substituted 1,4-distyrylbenzenes.

3% by weight, based on the weight of the monomers, of the commercial optical brightener Lumogen Violet 570 (manufacturer: BASF) were added, as effect substances, to the emulsion mentioned in example 1a. After illuminating and separating, polymeric nanoparticles which are homogeneous and comprise the optical brightener are obtained. They can be used as additives for brightening in the plastics and paper industries.

3b) Preparation of Nanoparticles using Ultraphor SFGplus as Effect Substance

2% by weight, based on the weight of the monomers, of the commercial optical brightener Ultraphor SFGplus (manufacturer: BASF) are added as effect substance to the emulsion mentioned in example 1a, This is converted to a fine emulsion in a high-pressure emulsification process and, on exiting from the nozzle, is illuminated with UV.

After illuminating and separating, polymeric nanoparticles which are homogeneous and comprise the optical brightener are obtained.

3c) Preparation of Nanoparticles using Ultraphor RN as Effect Substance

1% by weight, based on the weight of the monomers, of the commercial optical brightener Ultraphor RN (manufacturer: BASF) is added, as effect substance, to the mixture of monomers and photoinitiator mentioned in example 1a. This is subjected at ambient temperature to a high-pressure emulsification process and, on exiting from the nozzle, illuminated with UV. After illuminating and separating, polymeric nanoparticles which are homogeneous and comprise the optical brightener are obtained.

### EXAMPLE 4

4a) Preparation of Nanoparticles using a UV Absorber

1.5% by weight, based on the weight of the monomers, of the commercial UV absorber Uvinul 3039 (UV absorber of cyanoacrylate type; manufacturer BASF) were added, as effect substance, to the emulsion mentioned in example 1a. After illuminating and separating, polymeric nanoparticles which are homogeneous and comprise the UV absorber were obtained. They can be used, e.g., as light-stability agents for varnishes and plastics or in the preparation of cosmetics.

4b) Preparation of nanoparticles using Uvinul 3008

2.0% by weight, based on the weight of the monomers, of the commercial UV absorber Uvinul 3008 (UV absorber of hydroxybenzophenone type; manufacturer BASF) are added, as effect substance, to the emulsion mentioned in example 1a. The fine emulsion was produced using ultrasound or high-pressure emulsification and, after illuminating and separating, polymeric nanoparticles which are homogeneous and comprise the UV absorber can be isolated. They can be used, e.g., in the preparation of cosmetics.

4c) Preparation of Nanoparticles using Uvinul 3008

8.0% by weight, based on the weight of the monomers, of the commercial UV absorber Uvinul 3008 (UV absorber of hydroxybenzophenone type; manufacturer BASF) are added, as effect substance, to the mixture of monomers and a conventional photoinitiator of the benzophenone type (1% by weight, based on the amount of monomers) mentioned in example 1a. The fine emulsion was produced by high-pressure pressure emulsification and, after illuminating and separating, polymeric nanoparticles which are homogeneous and comprise the UV absorber in the particles can be obtained.

4d) Preparation of Nanoparticies using Tinuvin P

3.0% by weight, based on the weight of the monomers, of the commercial UV absorber Tinuvin P (UV absorber of the benzotriazole type; manufacturer Ciba, Switzerland) are added, as effect substance, to the emulsion mentioned in example 1a.

The fine emulsion was produced using ultrasound or high-pressure emulsification and, after illuminating and separating, polymeric nanoparticles which are homogeneous and comprise the UV absorber can be isolated using standard processes. They can be used as light-stability agents.

### EXAMPLE 5

5a) Preparation of Nanoparticles with Addition of Epoxiconazole as Active Substance.

5% by weight, based on the weight of the monomers, of the commercial pesticide epoxiconazole (cereal fungicide, manufacturer BASF) are added, as active substance, to the mixture of monomers and photoinitiator mentioned in example 1a. After illuminating the fine emulsion produced and separating, polymeric nanoparticles which are homogeneous and comprise the pesticide in the particles can be obtained.

By addition of 0.5% by weight of Lumogen F Red as colorant and additionally of 5% by weight, based on the weight of the monomers, of the commercial pesticide epoxiconazole as active substance, nanoparticles can be prepared which are homogeneously colored and comprise the active substance in the polymer matrix.

5b) Preparation of Nanoparticles with Addition of a Biocide Component

4% by weight, based on the weight of the monomers, of the commercial biocide 2-methyl-2H-isothiazol-3-one are added, as active substance component, to the mixture of monomers and photoinitiator mentioned in example 1a. After illuminating the fine emulsion produced and separating, polymeric nanoparticles which are homogeneous and comprise the biocide in the particles can be obtained. 5c) Preparation of Nanoparticles with Addition of a Pharmaceutical Active Substance

5% by weight, based on the weight of the monomers, of the commercial pharmaceutical Ibuprofen are added, as active substance, to the mixture of monomers and photoinitiator mentioned in example 1a. After illuminating the fine emulsion produced and separating, polymeric nanoparticles which are homogeneous and comprise the pharmaceutical active substance in the particles can be obtained.

5d) Preparation of Nanoparticles with Addition of Vanillin

1% by weight, based on the weight of the monomers, of the commercial odorous substance vanillaidehyde is added, as active substance, to the mixture of monomers and photoinitiator mentioned in example 1a. After illuminating the fine emulsion produced and separating, polymeric nanoparticles are obtained which exhibit a marked smell of vanilla and which comprise the fragrance homogeneously distributed in the particles.

1-18. (canceled)

19. A process for the preparation of polymeric nanoparticles which comprises introducing one or more polymerizable monomers (M) and one or more dispersants (D) and/or one or more effect substances (E) and/or one or more active substances (A) into a nonsolvent (N) to form an emulsion and illuminating said emulsion with a suitable light source (L), resulting in a photopolymerization of the monomers (M).

20. The process according to claim 19, wherein at least two different monomers (M) are used.

21. The process according to claim 19, wherein the polymerizable monomers (M) are first mixed with a photoinitiator (P) and optionally one or more effect substances (E) and/or one or more active substances (A), an emulsion is subsequently produced from this mixture, together with the nonsolvent (N) and the dispersant (D), and the emulsion thus formed, optionally after producing a fine emulsion produced by introducing shear energy, is subjected to an illuminating stage with UV light.

22. The process according to claim 19, wherein said monomer is a polymerizable acrylate monomer.

23. The process according to claim 19, wherein the comprises introducing one or more polymerizable monomers (M) and one or more dispersants (D) and one or more effect substances (E) into a nonsolvent (N) to form an emulsion and illuminating said emulsion with a suitable light source (L), resulting in a photopolymerization of the monomers (M).

24. The process according to claim 19, wherein the comprises introducing one or more polymerizable monomers (M) and one or more dispersants (D) and one or more active substances (A) into a nonsolvent (N) to form an emulsion and illuminating said emulsion with a suitable light source (L), resulting in a photopolymerization of the monomers (M).

25. The process according to claim 22, which further comprises an effect substance (E) and/or an active substance (A), of one or more photoinitiators (F).

26. The process according to claim 19, wherein use is made of an emulsion which additionally comprises one or more effect substances (E) selected from the group consisting of dyes, optical brighteners, UV absorbers and pigments and/or one or more active substances (A) selected from the group consisting of pesticides, biocides, pharmaceuticals and fragrances.

27. The process according to claim 19, wherein a preemulsion is first produced by stirring and this is then treated by a process for fine emulsification with the introduction of shear energy and subsequently illuminated with a suitable light source and photopolymerized.

28. The process according to claim 19, wherein a preemulsion is first produced and this is then treated by a continuous process for fine emulsification with introduction of shear energy, the emulsified particles comprising the monomers achieving a mean size of less than 4 μm, and subsequently illuminated with a UV light source and photopolymerized.

29. The process according to claim 19, which is carried out at a temperature of 10 to 50° C.

30. The process according to claim 19 wherein a preemulsion is first produced and this is then converted to a fine emulsion using a rotor/stator process or using a high-pressure homogenization process and the fine emulsion is illuminated with a UV light source and photopolymerized.

31. Polymeric nanoparticles which are prepared by the process of claim 19.

32. The polymeric nanoparticles according to claim 31, which comprise one or more effect substances (E) and/or one or more active substances (A).

33. The polymeric nanoparticles according to claim 31, which exhibit a mean particle size of less than 4 μm.

34. The polymeric nanoparticles according to claim 31, which comprise at least one polyacrylate and which exhibit a mean particle size of 0.05 μm to 3.0 μm.

35. The polymeric nanoparticles according to claim 31, which comprise at least one effect substance (E) selected from the group consisting of dyes, optical brighteners, UV absorbers and pigments.

36. The polymeric nanoparticles according to claim 31, which comprise at least one active substance (A) selected from the group consisting of pesticides, biocides, pharmaceuticals and fragrances.

37. The polymeric nanoparticles according to claim 31, which comprise at least one active substance (A) selected from the group consisting of pesticides, biocides, pharmaceuticals and fragrances and in addition at least one effect substance (E) selected from the group consisting of dyes, optical brighteners, UV absorbers and pigments.
